(19) United States
(12) Patent Application Publication
(10) Pub. No.: US 2004/0115614 A1
(43) Pub. Date: Jun. 17, 2004

(54) Electrophysiology assay methods

(76) Inventors: Paul Burnett, Somerville, NJ (US); Janet Kay Robertson, Easton, PA (US); Robert Allan Zivin, Skillman, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/634,692

(22) Filed: Aug. 5, 2003

Related U.S. Application Data
(60) Provisional application No. 60/401,156, filed on Aug. 5, 2002.
(60) Provisional application No. 60/434,917, filed on Dec. 20, 2002.

(51) Int. Cl. C12Q 1/00

(52) U.S. Cl. 435/4

(57) ABSTRACT

The present invention provides a system that is capable of supplying electric field stimulation to a cell and optically monitoring a physiological response of the stimulated cell. The invention also provides methods of eliciting a physiological response in a cell or characterizing the biological activity of a candidate compound using an electrical field stimulation (EFS) device. Such methods are readily amenable to high throughput screen (HTS).

This patent application claims priority from U.S. Provisional Patent Application 60/401156 filed Aug. 5, 2002 and U.S. Provisional Patent Application 60/434,917 filed Dec. 20, 2002, both entitled “An Electrophysiology Assay Method”, the contents of which are incorporated herein in their entirety.

### FIELD OF THE INVENTION

The present invention relates to electrophysiological assays. In particular, the present invention provides methods of eliciting a physiological response in a cell or characterizing the biological activity of a candidate compound using an electrical field stimulation (EFS) device.

### BACKGROUND OF THE INVENTION

Voltage-gated ion channels determine, in part, the electrical activity of neuronal and muscle cells. In addition, these channels participate in the secretion of neurotransmitters and hormones. Due to their implication in a wide variety of diseases, such as cardiovascular, CNS or metabolic diseases, the channels are emerging as a target class of increasing importance to the pharmaceutical industry. Although different families of voltage-gated ion channels have different structures, they share common functional elements. For example, the channels are trans-membrane proteins with an ion-selective pore. Channel gating is controlled by a voltage sensitive region of the protein containing charged amino acids that allow conformational changes of the protein in response to changing membrane potential.

Identifying a new drug which has specific modulatory effects on voltage-gated ion channels is complicated by the fact that unlike other channels, such as G protein coupled receptors (GPCRs) or ligand-gated ion channels, voltage-gated ion channel activity is evoked by changes in plasma membrane potential rather than agonist binding. Assays to screen for modulators of voltage-gated ion channel require both methods of stimulating and detecting the plasma membrane potential of cells.

The existing technologies for identifying a modulator for a voltage-gated ion channel are a compromise between throughput, physiological relevance, sensitivity and robustness. The best-known assay today is probably the patch-clamp assay. The patch-clamp technique controls the electrical potential difference across a small patch of membrane or across the plasma membrane of an entire cell and directly assesses the current carried by ions crossing the membrane at that voltage through ionic channels. This technology provides high quality and physiologically relevant data of ion-channel function at the single cell or single channel (within a small patch of membrane) level. But, setting up patch-clamping experiments is a complicated process requiring highly trained personnel because the system is vulnerable to interference from vibration and electrical noise. The throughput of patch-clamp technicians is, at best, 10-30 data points per day (Xu, et al. (2001), Drug Discovery Today, 6:1278-12887). Such low throughput and high labor-cost is far from acceptable for high throughput screen (HTS) purposes. Although several companies are attempting to automate the patch-clamp process, the current complexity and reproducibility of the experimental setup renders it unsuitable for an HTS application.

Optical recording using voltage sensitive dyes became popular because of the significantly greater throughput for screening applications (currently, up to 100,000 compounds per day) and the highly sensitive analysis of transmembrane potential (Xu, et al. (2001), Drug Discovery Today, 6:1278-12887). Such methods do not directly measure ionic current but measure either membrane-potential-dependent or ion-concentration-dependent changes of optical signals, such as fluorescent signals from fluorescent dyes loaded into the cytosol or cell membrane, as a result of ionic flux. Compared to the use of a patch-clamp, optical analytic methods do not inherently permit the plasma membrane potential of a cell to be regulated. These methods frequently require pharmacological intervention to activate the channels under investigation, leading to the possibility that artifacts can be introduced or false hits generated (Denyer, et al., (1998), Drug Discovery Today, 3:323-332).

US2002/0025568 describes a method of characterizing the biological activity of a candidate compound that includes placing one or more cells into an area of observation in a sample well; exposing the cells to a compound; repetitively exposing the cells to a series of biphasic electric fields at a rate of approximately 20 to 100 pulses per second, wherein the electric fields exhibit limited spatial variation in intensity in the area of observation of less than about 25% from a mean intensity in that area, and wherein the electric fields produce a controlled change in transmembrane potential of the cells; and monitoring changes in the transmembrane potential of the cells by detecting fluorescence emission of a FRET based voltage sensor from an area of observation containing the cells cells. The method uses an assembly that is described in US2002/0025573. In such an assembly, electrical fields are applied via a pair of substantially parallel electrodes that are either dipped into the sample wells with or without satellite electrodes, or plated onto the bottom surface of sample wells. Optical monitoring is limited to a clear line of sight through the bottom surface of the sample well that is not covered by electrodes.

Metal microelectrodes patterned on glass substrates have been used to stimulate cells and record the subsequent electrical response (Thomas, et al., (1972), Experimental Cell Research. 74:61-66; Pine, et al., (1980), Journal of Neuroscience Methods. 2:19-31). Microelectrodes fabricated from a transparent metal oxide, indium tin oxide (ITO), have also been used to stimulate cells and record the subsequent electrical response (Gross, et al., (1985), Journal of Neuroscience Methods, 15:243-252). In this work, the tips of the microelectrodes were coated with platinum black (which is opaque) to reduce the impedance of the microelectrode and facilitate data recording by increasing the signal-to-noise ratio. But the platinum black coating is not necessary for electrically stimulating the cells. Because of the coating, these microelectrodes were not wholly transparent and were not optimized for uniform stimulation of multiple cells.

US2003/0018360 claims an electrical field stimulation (EFS) device for stimulating cultured cells. The device includes a transparent substrate, an insulator plate secured adjacent to the transparent substrate having at least one well formed therethrough for containing the cultured cells, a surface of the transparent substrate defining the floor of the well, a first transparent electrode disposed on the surface of the transparent substrate for covering at least a portion of the floor, and a second electrode in electrical communication with the first transparent electrode.

The present invention provides a new assay method that supplies electric field stimulation to a cell to elicit a physiological response and is compatible with optical recording of the physiological response in the stimulated cell. This assay is readily amenable to HTS.

### SUMMARY OF THE INVENTION

In one aspect, the present invention relates to a method of measuring a physiological response in a cell, comprising the steps of: introducing one or more cells in a liquid medium into a well of an electric field stimulation device, wherein the device comprising at least one transparent electrode disposed on the surface of the transparent bottom of the well; labeling the cell with an optically detectable marker; exposing the cell to repetitive electric pulses supplied by the transparent electrode and a second electrode of opposing polarity, wherein the repetitive electric pulses are of about between 250-1000 μs duration at about 1-100 pulses/s and about 2-120 V amplitude, wherein the electric pulses produce a controlled change in a physiological response of the cell; and detecting an optical signal associated with the optically detectable marker. The method further comprises comparing the optical signal with an optical signal measured from a cell that is not exposed to the repetitive electric pulses. Preferably, the physiological response in a cell comprises a change in the activity of an ion channel, a change in the secretion or absorption of a biological molecule by the cell, plasma membrane rearrangement, intracellular rearrangement, a change in cellular metabolism, apoptosis, or gene transcription.

The invention further relates to a method of characterizing the biological activity of a candidate compound, comprising the steps of: introducing one or more cells in a liquid medium into a well of an electric field stimulation device, wherein the device comprising at least one transparent electrode disposed on the surface of the transparent bottom of the well; labeling the cell with an optically detectable marker; contacting the cell with a test compound; exposing the cell to repetitive electric pulses supplied by the transparent electrode and a second electrode of opposing polarity, wherein said repetitive electric pulses are of about 250 to about 1000 μs duration at about 1 to about 100 pulses/s and about 2 to about 120 V amplitude, to produce a controlled change in a physiological response of the cell; detecting an optical signal associated with the optically detectable marker; and comparing the optical signal with an optical signal measured from a cell that is not contacted with the candidate compound. Preferably the transparent electrode includes an electrically conductive transparent material or is a metallic optically transparent electrode. In one embodiment the electrically conductive transparent material is selected from a group consisting of indium tin oxide (ITO), zinc oxide (ZnO), SnO2, CdO, MgIn2O4, Al-doped ZnO film, a diamond thin film, and a combination thereof. The transparent electrode can further include a layer of an insulating transparent material external to the electrically conductive transparent material. The insulating transparent material is preferably a transparent dielectric, selected from silicon dioxide (SiO2), silicon nitride (Si3N4), and silicon oxynitride (SiOxNy) and the thickness of the insulating transparent material is about 100 Å to about 2000 Å. In one embodiment of this aspect, a second electrode of opposing polarity is inserted into the fluid bathing the cells inside the well, wherein a voltage applied between the transparent electrode and the second electrode creates a vertical electric field capable of stimulating cells inside the well. Preferably the second electrode of opposing polarity comprises an electrically conductive transparent material or is a metallic optically transparent electrode and may further comprise an electrically conductive non-transparent material. Preferred non-transparent material include gold, platinum, palladium, chromium, molybdenum, iridium, tungsten, tantalum, titanium, stainless steel, carbon, graphite and polypyrrole. In one preferred embodiment two transparent electrodes are fabricated to contain interdigitated fingers covering the surface of the transparent bottom of the well. A preferred width and spacing is provided such that a single cell can contact at least two or more electrodes of opposing polarity.

In this aspect of this preferred embodiment, the physiological response is a change in the conductivity of an ion channel wherein the ion channel is selected from the group consisting of a potassium channel, a calcium channel, a chloride channel, a sodium channel, a non-specific ion channels, and a combination thereof. Preferred optically detectable markers include a fluorescent dye, a radioactive ion, a fluorescent protein, a luminescent protein, a protein tagged with a fluorescent or luminescent epitope, a change in the refractive index of the cells, or a voltage sensor selected from the group consisting of FRET based voltage sensors, electrochromic transmembrane potential dyes, transmembrane potential redistribution dyes, radioactive ions, ion sensitive fluorescent or luminescent dyes, and ion sensitive fluorescent or luminescent proteins. The optical signal associated with the optically detectable marker is preferably monitored via an imaging system and preferred imaging systems include a microscope connected to a charge-coupled device camera, a photodiode array, or a photomultiplier tube. Preferred imaging system are comprised of a plate reader, connected to a charge-coupled device camera, a photodiode array, or a photomultiplier tube. Repetitive electric pulses are preferably supplied in a square wave-form, a sinusoidal wave-form, or a saw tooth wave-form.

In yet another preferred embodiment, the repetitive electric pulses used to stimulate cells are in the form of a square wave, at about 750 μs per pulse duration, and 8 pulses/s for 3 s.

The invention further relates to a system for supplying electric field stimulation to a cell and optically monitoring a physiological response of the stimulated cell, comprising: an electric field stimulation device comprising a well and an transparent electrode disposed on the surface of the transparent bottom of the well; a cell labeled with an optically detectable marker placed and its bathing fluid within the well of the electric field stimulation device; a means for providing electrical stimulation; and an imaging system for detecting the optical signal from the cell.

Other aspects, features and advantages of the invention will be apparent from the following disclosure, including the detailed description of the invention and its preferred embodiments and the appended claims.

### BRIEF DESCRIPTION OF THE DRAWINGS

FIG. 1 shows the cross-section of an EFS device during fabrication. The drawing is not to scale. The vertical scale has been expanded for clarity.

FIG. 2a shows the top view of an EFS device including two electrode configurations while FIG. 2b is a schematic view of a well containing two inter-digitated electrodes.

FIG. 2c is a magnified partial view of the well of FIG. 2b containing two inter-digitated electrodes. The drawing is not to scale. In this embodiment the inter-digitated fingers are preferably about 5 μm wide and 5 μm apart. The well containing these electrodes is about 3500 μm in diameter. The entire bottom surface of the well is covered with interdigitated fingers of Indium Tin Oxide (ITO).

FIG. 3 illustrates changes in plasma membrane potential of SK-N-SH cells with electrical stimuli supplied by the prototype EFS device. Figures show plasma membrane potential changes from SK-N-SH cells (human neuroblastoma cells) cultured in a well containing interdigitated electrodes (FIG. 3a) or Ag/AgCl electrode (FIG. 3b). Cells were stimulated at the times and conditions indicated by arrows. Each stimulus consisted of a train of 750 μs pulses (8 pulses per s, for 3 s). Data shown are the representative mean changes in fluorescence intensity, corresponding to changes in membrane potential (100 cells per experiment). The x-axis: time (seconds); the Y-axis: voltage-induced changes in fluorescence intensity (detected at 510 nm) of the fluorescent probe following excitation with light of wavelength 488 nm.

FIG. 4 illustrates voltage-induced changes in plasma membrane potential of SK-N-SH cells in the presence of TTX, a specific inhibitor for voltage-gated Na⁺ channels (A). The effect of 100 nM TTX was also investigated (B). Electrical stimuli were applied to SK-N-SH cells cultured on an EFS device at the voltages, conditions and times indicated by arrows. Each stimulus consisted of a train of 750 μs pulses (8 pulses per s, for 3 s). Data shown were representative mean responses from all cells in one experimental preparation, using wells with inter-digitated electrodes. Results are expressed as a percentage of the maximal depolarization observed with 50 mM KCl (100 cells per preparation).

FIG. 5 shows the stimulus-depolarization relationship of SK-N-SH cells stimulated on an EFS device. Cells were pre-incubated with TTX at the concentrations shown, for 30 min prior to experimental manipulation. Data shown are the mean of three separate experimental preparations (100 cells per preparation). Panel (a) shows the effect of stimulus amplitude on the rate of depolarization following electric field stimulation: solid square: control; solid triangle: 1 nM TTX; solid diamond: 10 nM TTX; solid circle: 100 nM TTX; open square: 1 μM TTX; and open triangle: 10 μM TTX. Panel (b) demonstrates the effect of TTX on the membrane potential responses elicited by the indicated stimulus amplitudes: solid square: 2 V; solid triangle with up-arrow head: 4V; solid triangle with down-arrow head: 6 V; solid diamond: 8 V; solid circle: 10 V; open square: 12 V.

FIG. 6 shows voltage-induced changes in plasma membrane potential of HEK cells stably expressing hERG channels—a voltage-gated K⁺ channel, cultured in a well containing interdigitated electrodes (A). Voltage stimuli were applied to an EFS device at the amplitudes and times shown and changes in the plasma membrane potential were recorded. The effect of pre-incubation with 1 μM cisapride (30 min), an inhibitor for hERG channels, was also investigated (B). Data shown are the mean values obtained from 1 well (150 cells per well).

FIG. 7 shows voltage-induced changes in the plasma membrane potential of HEK cells stably expressing hERG channels cultured in a well containing an Ag/AgCl electrode. Voltage stimuli were applied to the EFS device at the amplitudes and times shown and changes in the plasma membrane potential were recorded. Data shown are the mean values obtained from 1 well (150 cells per well).

FIG. 8 shows the effect of extracellular Na⁺ removal on voltage-induced changes in the plasma membrane potential of HEK cells stably expressing hERG channels cultured in a well containing interdigitated electrodes. Voltage stimuli were applied to an EFS device at the amplitudes and times shown and changes in the plasma membrane potential were recorded. Data shown were the mean values obtained from 1 well (150 cells per well).

### DETAILED DESCRIPTION OF THE INVENTION

All publications cited herein are hereby incorporated by reference. Unless defined otherwise, all technical and scientific terms used herein have the same meaning as commonly understood to one of ordinary skill in the art to which this invention pertains.

The following are abbreviations that are at times used in this specification below:

AC=alternating current

ATCC=American Type Culture Collection

CCD=charge-coupled device

CNS=central nervous system

DC=Direct Current

ECACC=European collection of cell cultures

EFS=electrical field stimulation

FLIPR=Fluorimetric Imaging Plate Reader

GFP=green fluorescent protein

HTS=high throughput screen

IDF=interdigitated fingers

ITO=indium tin oxide

FRET=fluorescence resonance energy transfer

s=second

V=volt

As used herein, the terms “comprising”, “containing”, “including” and “having” are used in their open, non-limiting sense.

A “cell” refers to any type of cell, including eukaryotic cells such as animal cells, plant cells, insect cells, yeast cells, and prokaryotic cells such as bacterial cells. The term includes tissue culture cell lines that can be relatively easily grown and transfected with high efficiency. Many cell lines are commercially available through the American Type Culture Collection (ATCC, http://www.atcc.org), as well as the European collection of cell cultures (ECACC, http://www.camr.org.uk). The term also includes primary cell lines, or cells within tissue slices.

The term “depolarize” means to cause the transmembrane potential of a cell to become closer to zero. In the case of cells that are normally at negative resting potentials, this term means that the transmembrane potential changes in a positive direction.

An “electric field” refers to a patch of space that causes the acceleration of electric charges located at that patch of space. The electric field can provide an electrical stimulation to cells located within the field, initiating physiological responses of the cells. The electric field can be supplied to a cell within the field in the form of repetitive electric pulses. The term “repetitive” means to repeat at least twice.

An “electric field stimulation (EFS)” device is a device capable of generating an electric field that can be used to electrically stimulate a living cell. In a preferred embodiment, the EFS device includes a transparent substrate, an insulator plate secured adjacent to the transparent substrate; at least one well formed through the insulator plate for culturing at least one cell, wherein a surface of the transparent substrate defining a floor of the well, a first transparent electrode disposed on the surface of the transparent substrate, wherein the transparent electrode covers at least a portion of the floor of the well; and a second electrode in electrical communication with the first transparent electrode. A voltage can be applied across the first transparent electrode and the second electrode to create an electric field that can electrically stimulate the cell cultured in the well of the EFS.

An “electrode” is a terminal that conducts an electric current into or away from various conducting substances in a circuit. It can be either an anode or a cathode. An “anode” is the electrode toward which electrons are drawn or to which an external positive voltage supply is connected. A “cathode” is the electrode toward which cations are drawn and to which an external negative voltage supply is connected.

A “fluorescent agent” refers to an agent capable of absorbing light and then re-emitting at least some fraction of that energy as light over time. The term “fluorescent agent” includes discrete compounds such as a fluorescent dye, fluorescent proteins, and macro-molecular complexes. The term also includes molecules that exhibit long-lived fluorescence decay such as lanthanide ions and lanthanide complexes with organic ligand sensitizers, which absorb light and then re-emit the energy over milliseconds.

The term “fluorescent protein” refers to a protein capable of forming a fluorescent, intrinsic chromophore either through the cyclization and oxidation of internal amino acids within the protein or via the enzymatic addition of a fluorescent co-factor. The term includes, but is not limited to, wild-type fluorescent proteins such as the green fluorescent protein (GFP) from Aequorea victoria, and engineered mutants that exhibit altered spectral or physical properties. The term also includes recombinant proteins comprising a fluorescent epitope tag, which, for example, can be made of a chromophore. The term does not include proteins that exhibit weak fluorescence by virtue only of the fluorescence contribution of non-modified tyrosine, tryptophan, histidine and phenylalanine groups within the protein.

The term “FRET” refers to fluorescence resonance energy transfer. For the purposes of this invention, “FRET” includes, but is not limited to, energy transfer processes that occur between two fluorescent agents, a fluorescent agent and a non-fluorescent agent, a luminescent agent and a fluorescent agent, and a luminescent agent with a non-fluorescent agent.

The term “high throughput screen (HTS)” refers to an assay design that allows easy screening of multiple samples simultaneously, and has the capacity for robotic manipulation. The “high throughput” assays are often optimized to reduce reagent usage, or minimize the number of manipulations in order to achieve the analysis desired. Examples of high throughput assay formats include 96-well or 384-well plates and “lab on a chip” microchannel chips used for liquid handling experiments.

The term “hyperpolarize” means to cause the transmembrane potential of a cell to move farther away from zero. In the case of cells that are normally at negative resting potentials, this term means that the transmembrane potential changes in a negative direction.

The term “luminescent agent” refers to an agent capable of absorbing energy, such as electrical (e.g. electro-luminescence), chemical (e.g. chemi-luminescence) or acoustic energy and then emitting at least some fraction of that energy as light over time. The term includes discrete compounds, molecules, naturally luminescent proteins, recombinant proteins comprising a luminescent epitope tag, and marco-molecular complexes. Examples of “luminescent agent” include the luminescent protein, such as the luciferase protein isolated from fire-flies or bacteria.

“Membrane potential” or “transmembrane potential” is the electrical potential difference across a plasma membrane, which is the external lipid bilayer membrane of a cell. Usually, the inner face of the plasma membrane has more negative electrical potential with respect to the outer face. The membrane potential can be an indicator of a cell's health and energy status. Changes in membrane potential of a cell can control the activity of a voltage-gated ion channel.

The term “multiwell plate” refers to a two dimensional array of addressable wells located on a substantially flat surface. Multiwell plates may comprise any number of discrete addressable wells, and comprise addressable wells of any width or depth. Common examples of multiwell plates include 96 well plates, 384 well plates and 1536 well plates.

An “optically detectable marker” or “optical marker” is an agent that can be detected by a means of optical detection. Such an agent can be a fluorescent agent, a luminescent agent, or a radioactive agent. Alternatively, an “optically detectable marker” can cause a change in refractive index in the sample under observation, and such a marker can be detected using polarized light.

A “transparent electrode” is an electrode that is optically transparent. Examples of “transparent electrodes” include, electrodes made of metal oxide transparent materials such as indium tin oxide (ITO), zinc oxide (ZnO), SnO₂, CdO, MgIn₂O₄, Al-doped ZnO film, or a combination of these materials. “Transparent electrodes” also include metallic optically transparent electrodes (Janssen et al., 1983, Surface Technology 20(1): 41-9), such as a platinum minigrid optically transparent electrode. “Transparent electrodes” further include electrodes made of other materials that are electrically conductive and transparent, such as a diamond thin film (Zak et al., 2001, Anal Chem. 73(5): 908-14).

A “voltage-gated ion channel” is a class of ion channels, whose state of activation changes or whose ion-selective pore opens or closes, in response to change in the electrical potential across the plasma membrane of the cell. As used herein, the “voltage-gated ion channel” is selected from the group consisting of a potassium channel, a calcium channel, a chloride channel, a sodium channel, a non-specific ion channels such as the VR1 receptor, or any biologically relevant combination of the above channels.

The term “voltage sensor” refers to an agent capable of sensing the changes of voltage and providing an indication of the transmembrane potential in a biological system. Examples of “voltage sensor” include, but are not limited to, FRET based voltage sensors, electrochromic transmembrane potential dyes, transmembrane potential redistribution dyes, extracellular electrodes, field effect transistors, radioactive ions, ion sensitive fluorescent or luminescent dyes, and ion sensitive fluorescent or luminescent proteins.

One general aspect of the present invention is to separate means of recording from that of stimulating. Thus, the present invention provides a method to electrically stimulate a cell using at least one transparent microelectrode and to record physiological responses of the stimulated cell using a means of optical imaging. The physiological responses that may be influenced by an electrical stimulation include, but are not limited to, the activity of an ion channel, the secretion or absorption of a biological molecule by the cell, plasma membrane rearrangement, intracellular rearrangement, cellular metabolism, apoptosis, and gene transcription.

According to the present invention, cells of interest are first placed in a liquid medium into one or more wells of an EFS device. The liquid medium that is used in this invention includes any of a variety of cell media or buffers that support the integrity of the cells in culture, including a variety of growth mediums, balanced salt solutions and buffered salines. The EFS device preferably comprises at least one transparent electrode disposed on the surface of the transparent bottom of the well. The transparent surface electrode can have a range of sizes and surface area. It can cover the whole surface of the bottom of the well, or just a portion of the surface. For HTS, the transparent electrode can also be fabricated across the entire bottom of a multi-well plate, such as a 96 well plate, a 384 well plate, or a 1536 well plate.

The transparent electrodes of this invention can be made from any electrically conductive transparent material known in the art. Some examples of transparent electrodes that can be used in the invention are described supra. In a preferred embodiment, the method of the invention uses a transparent electrode comprising a metal oxide ITO. Methods for making transparent electrodes are known to those skilled in the art (see Lu et al., 2002, Qingdao Huagong Xueyuan Xuebao, 23(1): 15-18; Saijo et al., 2001, Kagaku to Kogyo, 75(8): 368-372; Liu, 1999, Cailiao Kexue Yu Gongcheng, 17(2): 98-100; and Janssen et al. 1983, supra). Example 1 illustrates a preferred process for fabricating a surface transparent electrode.

In a preferred embodiment, the transparent electrode further comprises a layer of insulating transparent material at an effective thickness outside the electrically conductive transparent material such as ITO. This thin layer protects the electrode by chemically isolating the conductive transparent material from the bathing fluid of the cell that the electrode is exposed to. This thin layer also prevents the coupling of direct current (DC) to the system and thus reduces joule heating of the solution. This layer also isolates cells from the conductive transparent material. Examples of such insulating transparent materials are transparent dielectrics, including, but not limited to, silicon dioxide(SiO₂), silicon nitride (Si₃N₄), silicon oxynitride (SiOₓN_{y}), and the like. The effective thickness of the layer is that at which it is thick enough to prevent electrical breakdown of the insulating transparent material, and it is thin enough to couple an alternating current (AC) or a time variant current to the cell. Preferably, the effective thickness of the layer of the insulating transparent material is about 100 Å to 2000 Å. For example, a thin layer of silicon dioxide (SiO₂) coated outside the ITO can prevent ITO darkening. At an electric field strength of 6×10⁶ V/cm, a thickness of about 333 Å of the layer of insulating transparent material is required in order for SiO₂ to withstand 20V electric field stimulation without experiencing breakdown.

Cells can be cultivated in a well of an EFS device for a relatively long period of incubation time, or transferred to the well of the EFS device shortly prior to the assay. The bathing fluid of the cells can be the growth medium for the cell or any suitable buffer solution. Preferably, cells attach to the bottom surface of the well where the transparent electrode is deposed. Some types of cell readily attach to the surface without additional manipulation. Other types, however, require pre-coating the surface with a factor to promote cell attachment. Such factors include, but are not limited to, poly-d-lysine, poly-l-lysine, collagen, or laminin, heparin sulphate proteoglycan, fibronectin, vitronectin, gelatin, or poly-l-ornithine. The ability of these factors to promote cell attachment will have to be examined individually for each cell type to be tested. The effect of the electrode substrate on cell viability can be examined using standard assays, such as trypan blue exclusion, or plate reader-based assays, where metabolic conversion of probes (MTT or Alamar blue) are used to determine cell viability. Cells cultured inside wells of the EFS device are compared to parallel populations of cells cultured under conventional laboratory conditions.

In one embodiment, the cells used in the assay are cell lines containing an endogenous voltage-gated ion channel, such as a pulmonary artery smooth muscle cells (PASMC), mammalian cardiac cells, or human neuroblastoma cells. Examples of such cell lines include, but are not limited to, SK-N-SH, HEK-293 cells, RBL cells, F11 cells, or HL5 cells. In another embodiment, the cell is a recombinant cell line containing a recombinant voltage-gated ion channel or subunit thereof.

Preferably, the cell or cells used can be a cell line that has no or very low detectable endogenous expression of other ion channels, such as CHO-K1, CHL, or LTK(−) cell lines. These cells inherently have a resting potential above the activation and inactivation thresholds of most voltage-gated channels. An exogenous ion channel expressed in such a host cell can be a major modulator of transmembrane potential. Thus, the activity of the exogenous ion channel can be easily and unambiguously monitored. Standard molecular biology techniques can be used to generate a recombinant expression vector, such as a plasmid, that carries the nucleotide sequence encoding the exogenous ion channel. Such an expression vector can be introduced into a desired host cell via conventional transformation or transfection techniques. As used herein, the terms “transformation” or “transfection” refers to a process by which cells take up foreign DNA and may or may not integrate that foreign DNA into their chromosome. Transfection can be accomplished, for example, by various techniques including calcium phosphate or calcium chloride co-precipitation, DEAE-dextran-mediated transfection, lipofection, or electroporation, protoplast fusion. Suitable methods for constructing a recombinant host cell can be found in Maniatis et al. (Molecular Cloning: A Laboratory Manual, Second Edition (Cold Spring Harbor Laboratory, Cold Spring Harbor, N.Y., 1989)), and other laboratory manuals.

Also preferred is a recombinant cell line comprising a voltage-gated ion channel and a second ion channel which helps to maintain the transmembrane potential of the cell below the threshold activation potential for the voltage-gated channel. For example, the resting membrane potential of certain cell lines (such as CHO, tsA201, or HEK293 cells) is low (at around −40 mV). Within such cells, some voltage-gated ion channels undergo voltage dependent steady state inactivation and require a more negative membrane potential to shift the channels into closed conformation states from which the channels can be activated. Activation of a co-expressed second ion channel, such as a small conductance Ca²⁺-activated K⁺ (SK) channel, may cause hyperpolarization and result in more negative membrane potential, allowing subsequent activation of the voltage-gated ion channel.

According to the present invention, cells used in the assays of this invention are labeled with an optically detectable marker. Depending on the type of physiological responses to be studied upon electrical stimulation, any type of optical markers described supra can be used to label the cells. In order to monitor membrane potential or ion channel conductivity, cells can be labeled with voltage sensors or ion sensitive dyes, or molecules, that typically exhibit a change in their optical property, such as fluorescent or luminescent characteristics, as a result of changes in membrane potential or ion channel conductivity. For example, the fluorescent Ca²⁺ indicator dyes such as fluo-3 or fura-2, Na⁺ indicator SBFI, or a voltage-sensing, lipophilic, fluorescent oxonol dye can be used to monitor changes in membrane potential or ion channel conductivity. Alternatively, radio-labeled quanidinium can be used for monitoring Na⁺ channels, and ⁸⁶Rb for K⁺ channels. These radioactive ions or non-toxic dyes can be loaded into most cell types.

In a preferred embodiment, a method of FRET can be used to monitor membrane potential or ion channel conductivity (see U.S. Pat. No. 5,661,035). Cells are labeled with two reagents that undergo energy transfer to provide a ratiometric optic readout that is dependent upon the transmembrane potential. For example, cells can be labeled with a voltage sensing lipophilic dye and a voltage insensitive fluorophore capable of being associated with a cell membrane (See Gonzalez et al., 1999, Drug Discovery Today, 4:431-439). FRET can also be used for optically monitoring other biological activities, such as protein-protein interaction, etc.

In another embodiment, optical markers such as a fluorescent or luminescent protein, a fluorescently labeled small molecule precursor to a secreted substance, or a fluorescently labeled nucleic acid molecule, can be used to optically monitor physiological responses such as the secretion or absorption of a biological molecule by the cell, plasma membrane rearrangement, intracellular rearrangement, cellular respiration, apoptosis, and gene transcription. For example, movement of a secretory granule and release of granule cargo during a single exocytotic/secretory event, have been successfully optically monitored using these optically detectable marker molecules (Maskos et al., (2002), PNAS, U.S.A., 99:10120-5). Preferably, the fluorescent protein is selected from the group consisting of a GFP and its mutant derivatives, reef coral fluorescent proteins and their mutant derivatives, a FRET based sensor of ions, and a fluorescent pH-sensitive indicator including carboxy-SNARF. The luminescent protein can be an ion-sensitive indicator, for example, the aequorin (Mitchell, el al.,(2001), Journal of Cell Biology. 155(1): 41-51).

Depending on the types of optical markers used to label the cells and the instrument used for optical detection, extra markers may or may not need to be washed out from the liquid bathing the cells prior to the optical analyses. For example, no washing is needed when voltage-sensitive membrane potential dye is used for FLIPR detection: depolarized cells are able to accumulate the dye to a greater extent than hyperpolarized cells, and the dye has low fluorescence in aqueous solution and much higher fluorescence when it binds to the hydrophobic plasma membrane.

According to the present invention, cells used in the assays of the present invention are subjected to repetitive electric pulses supplied by the transparent electrode and a second electrode of opposing polarity, wherein said repetitive electric pulses are of about 250-1000 μs duration at about 1-100 pulses/s and 2-120 V amplitude, and produce a controlled change in the physiological response of said cell.

In one embodiment, the second electrode of opposing polarity is inserted into the fluid bathing the cells inside the well. A voltage applied between the transparent surface electrode and the second electrode can create a vertical electric field capable of stimulating cells inside the well. For this configuration, the second electrode can be either transparent or non-transparent. A non-transparent electrode can be made from any non-transparent conductive material that is inert in saline. Such materials include, but are not limited to, noble metals (including gold, platinum, and palladium), refractory metals (including chromium, molybdenum, iridium, tungsten, tantalum, and titanium), corrosion-resistant alloys (including stainless steel), and carbon or other organic conductors (including graphite and polypyrrole).

In a preferred embodiment, the second electrode of opposing polarity is also transparent and integrally disposed on the same bottom surface of that containing the first transparent electrode. Preferably, the two electrodes are fabricated to contain interdigitated fingers (IDF), which can cover the entire surface of the bottom of the well. Many widths of electrode and their spacing are possible. Preferably, the interdigitated fingers from the two opposing electrodes are formed of such a width and have such a spacing between the adjacent fingers that a cell attached to the bottom surface of the well can contact two or more electrodes, resulting in very efficient stimulation of the cell. A voltage applied between these two transparent electrodes can create a horizontal electric field capable of stimulating cells inside the well. This electrode configuration removes the need for an electrode to be immersed into the well from above, and therefore enables easy access for fluidics and optical imaging.

The present invention uses repetitive electric pulses to stimulate a cell because it was shown previously that a current pulse passed through an electrode created a voltage gradient in the medium sufficient to depolarize nearby axons and cell bodies, causing them to fire action potentials (Regehr et al., (1989), J. Neuroscience methods, 30:91-106). In addition, it was shown previously that application of repetitive electrical stimulation pulses to the fluid bathing a cell modulated membrane potential of the cell that has at least one voltage-gated ion channel (US2002/0025573).

The electrical stimulation must be optimized to elicit a desired physiological response in the stimulated cell and to avoid killing or over heating of the cell. Parameters that need to be optimized for the repetitive electric pulses, include, but are not limited to, the type of individual pulses, the overall amplitude of individual pulses, the duration of individual pulses, the gap of successive pulses, the duration of the train of pulses, number of pulses in the train, and the use of multiple pulse trains. Most of these parameters depend on the electrode configuration used, and the type of cells and physiological responses to be analyzed. For example, as shown in Example 7 larger amplitude of the pulses was required to activate sodium channels of SK-N-SH cells when the vertical electrode configuration was used, compared to that when the IDF electrode configuration was used. However, similar amplitude of pulses was required to activate hERG channels (a voltage-gated K⁺ channel) in recombinant HEK cells (Example 9) for both vertical and IDF electrode configurations.

Any biphasic pulse such as a square wave-form, a sinusoidal wave-form, or a saw tooth wave-form, can be used for the invention. Preferably, the square wave-form is used.

In one embodiment, repetitive pulses of electric stimuli are supplied to the cell in microsecond duration. Sustained voltages can result in electrode breakdown and loss of light transmittance, for example ITO darkening (Gross et al., (1993), J. of Neuroscience Methods, 50:131-143). Preferably, repetitive pulses of electric stimuli are supplied to the cell in about 250-1000 μs per pulse at 1-100 pulses/s. Most preferably, repetitive pulses of electric stimuli are supplied to the cell in about 750 μs per pulse at 8 pulses per second, and the train of pulses lasts about 3 seconds.

In another embodiment, repetitive pulses of electric stimuli are supplied to the cell with an amplitude of about 2-120 V. When high voltage, often greater than 120 V, was used, cells detached from the surface of the electrode (often due to cell death) resulting in lower detectable optical signal. When low voltage, often less than 2 V, was used, no activation of any physiological response could be observed. Preferably, repetitive pulses of electric stimuli are supplied to the cell with an amplitude of about 20-100 V.

According to the invention, an optical signal associated with the optically detectable marker can be detected during or after the cell is electrically stimulated by the repetitive pulses. The use of transparent electrodes in the present invention enables easy optical detection of biological activities via optically detectable marker molecules. Any instrumentation that is capable of inducing and recording an optical signal, such as luminescent, fluorescence, or radiation, can be used in the method of the present invention.

In a preferred embodiment, a microscope-based detection system, such as Pathway-HT (Atto Bioscience, Rockville, Md.) can be used for optical recording. The microscope-based detection system has the advantage of recording changes in fluorescence both temporally (changes in membrane potential over time) and spatially (movement of cells, reorganization of plasma membrane or cell death). In addition, the microscope-based detection system also enables optical recording at a single cell level. In operation, the EFS device can be placed on the sample stage of the microscope with the wells uppermost. The microscope objective can be brought into position from below. The electrodes of the EFS device are then connected to an apparatus that is used to supply a range of electrical stimuli to the cells under microscopic observation, such as the Grass Telefactor S48 electrical stimulator via an SIU5 stimulus isolation unit (Grass-Telefactor/Astro-Med, Inc., West Warwick, R.I.). The microscopic images can be analyzed manually or via a computer.

In another preferred embodiment, a multi-well plate reader, preferable a specialized kinetic plate reader can be used for optical recording. Examples of multi-well plate readers include, but are not limited to, the TopCount plate reader (Packard) for luminescent recording or scintillation counting, the Fluorimetric Imaging Plate Reader (FLIPR; Molecular Devices, Sunnyvale, Calif.) for fluorescent recording, and the Fusion plate reader (Packard) for recording luminescent, fluorescent, or radiation signals. These readers can be equipped with integrated liquid handlers to introduce compounds or other reagents into the assay wells in order to initiate and observe effects on biological activity. For example, in an HTS assay for blockers of voltage-gated Ca²⁺ channels, cells loaded with optically detectable marker molecules are cultured into multi-well plates comprising at least one transparent electrode attached at the bottom of the plate, and are exposed to a repetitive electric pulses stimulation supplied by the transparent electrode and a second electrode of opposing polarity before or after exposing the cells to test compounds. Compounds that block the channels appear as wells demonstrating no Ca²⁺ response to the electric field stimulation. The most common formats use 96- or 384-well plates although higher densities are technically feasible. When integrated with other automation, these systems are capable of high-throughput screening of over 10,000 compounds per day.

To deduce the effect of electrical stimulation on a cell, the optical signal measured from cells stimulated with repetitive pulses of electric stimulation are compared to that measured from cells not subjected to the electric stimulation.

In another general aspect, the present invention provides a method to characterize the biological activity of a candidate compound, i.e., to identify a compound that inhibits or activates a physiological response of a cell that is influenced by an electrical stimulation. In operation, an electrical stimulation is applied to a cell to elicit a physiological response following the procedure described supra. The effect of a candidate compound is deduced by comparing the optical signal measured from cells subjected to both the electric stimulation and candidate compound with that from cells subjected to only the electric stimulation not the candidate compound. The test compound can be administered to the cell prior, after, or during the electrical stimulation. In a preferred embodiment, the invention provides a method to identify a compound that inhibits or activates the activity of a voltage-gated ion channel. In another preferred embodiment, the invention provides a method to identify a compound that inhibits or activates the process of secretion or absorption of a biological molecule, plasma membrane rearrangement, intracellular rearrangement, cellular metabolism, apoptosis, or gene transcription.

In yet another preferred embodiment, the invention provides a method to identify a compound that inhibits or activates a non-voltage gated ion channel, such as a ligand-gated channel or a second-messenger-gated channel. In the presence of a voltage-gated ion channel in the cell membrane, repetitive pulses electric filed stimulation can set the transmembrane potential over a relative wide range thereby enable the analysis of virtually any ion channel of the cell. For example, electric field stimulation is capable of setting the transmembrane potential of between about +10 to about +60 mV in a cell expressing a voltage-gated sodium channel. And, electric field stimulation is capable of setting the transmembrane potential of between about −90 to about −30 mV in a cell expressing a voltage-gated potassium channel.

The compound identification methods of the invention can be in conventional laboratory format or adapted for high throughput. It is well known by those in the art that as miniaturization of plastic molds and liquid handling devices are advanced, or as improved assay devices are designed, that greater numbers of samples may be performed using the design of the present invention.

Candidate compounds encompass numerous chemical classes, although typically they are organic compounds. Preferably, they are small organic compounds, i.e., those having a molecular weight of more than 50 yet less than about 2500. Candidate compounds comprise functional chemical groups necessary for structural interactions with polypeptides, and typically include at least an amine, carbonyl, hydroxyl or carboxyl group, preferably at least two of the functional chemical groups and more preferably at least three of the functional chemical groups. The candidate compounds can comprise cyclic carbon or heterocyclic structure and/or aromatic or polyaromatic structures substituted with one or more of the above-identified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound typically is a DNA or RNA molecule, although modified nucleic acids having non-natural bonds or subunits are also contemplated.

Candidate compounds are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including: biological libraries; spatially addressable parallel solid phase or solution phase libraries: synthetic library methods requiring deconvolution; the “one-bead one-compound” library method; and synthetic library methods using affinity chromatography selection (Lam (1997) Anticancer Drug Des. 12:145). Alternatively, libraries of natural compounds in the form of bacterial, fungal, plant and animal extracts are available or readily produced. Additionally, natural and synthetically produced libraries and compounds can be readily modified through conventional chemical, physical, and biochemical means.

Further, known pharmacological agents may be subjected to directed or random chemical modifications such as acylation, alkylation, esterification, amidification, etc. to produce structural analogs of the agents. Candidate compounds can be selected randomly or can be based on existing compounds that bind to and/or modulate the function of biological activity of interest. For example, a source of candidate agents can be libraries of molecules based on known activators or inhibitors for a voltage-gated ion channel of interest, in which the structure of the compound is changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions. One of ordinary skill in the art in the preparation of combinatorial libraries can readily prepare such libraries based on existing activators/inhibitors.

A variety of other reagents also can be included in the mixture. These include reagents such as salts, buffers, neutral proteins (e.g., albumin), detergents, etc. that may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

To further illustrate the invention, the following examples are provided.

### EXAMPLE 1

### Fabrication of the Test EFS (Electric Field Stimulation) Device

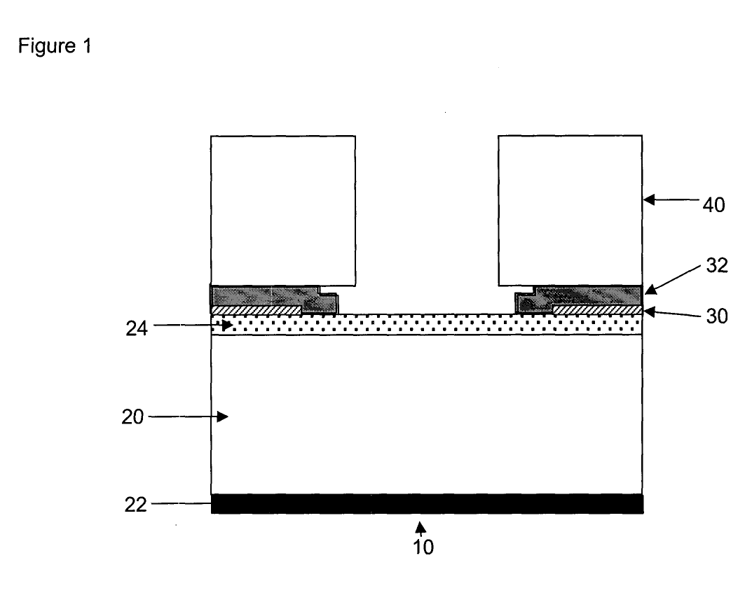

With particular reference to FIG. 1, a cross-section of an EFS device 10, fabrication process of the EFS device is illustrated. The EFS device was fabricated from a 600 μm thick Pyrex glass wafer 20. Aluminum 22 about 8000 Å thick was first deposited on the backside of the wafer to facilitate handling by automated equipment. The ITO 24, about 2700 Å thick, with a resistivity of about 25 Ω/square was coated to the frontside of the wafer and was subsequently patterned (for the interdigitated electrodes) using an ion mill. Next, Ti/Pt 30, about 100 Å/1200 Å thick, was evaporated and patterned using a liftoff procedure. The Ti/Pt reduced ohmic losses from the bond pads, where external electrical connection is made to the ITO in each well. A 5000 Å thick oxynitride (SiOₓN_{y}) layer 32 was then deposited by plasma-enhanced chemical vapor deposition. This insulating/passivation layer was patterned using a reactive ion etch. Removal of the backside aluminum 22 followed. An un-patterned layer of photoresist was spun onto the frontside of the wafer before the Al was removed to protect the frontside during the wet etch. After dicing, the photoresist was dissolved in acetone. The devices were then cleaned in isopropyl alcohol (IPA) followed by a thorough rinse in de-ionized (DI) water. O-rings or cloning wells 40 were then attached by hand to the device using a silicone adhesive. A second IPA/DI water clean completed the fabrication process.

### EXAMPLE 2

### EFS Device

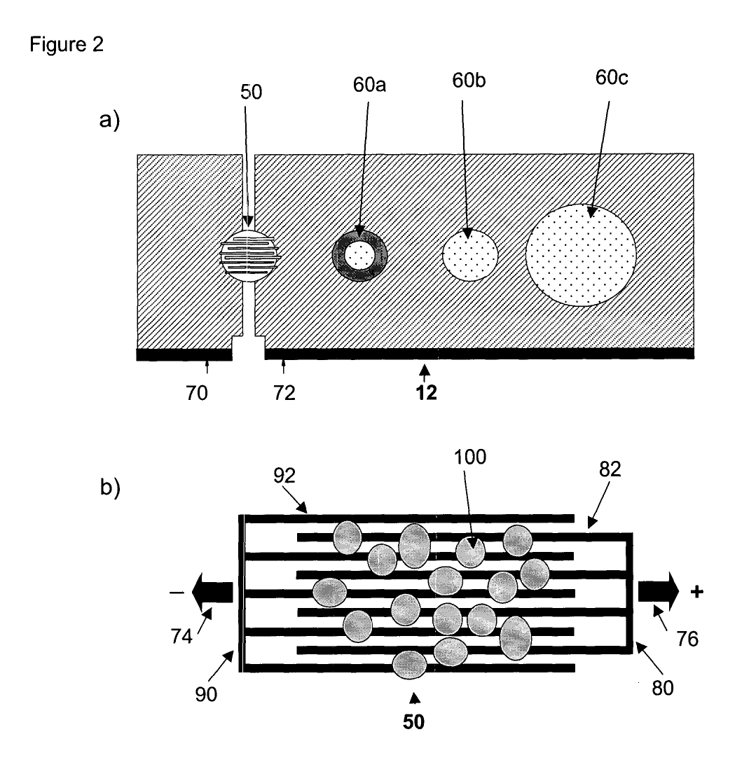

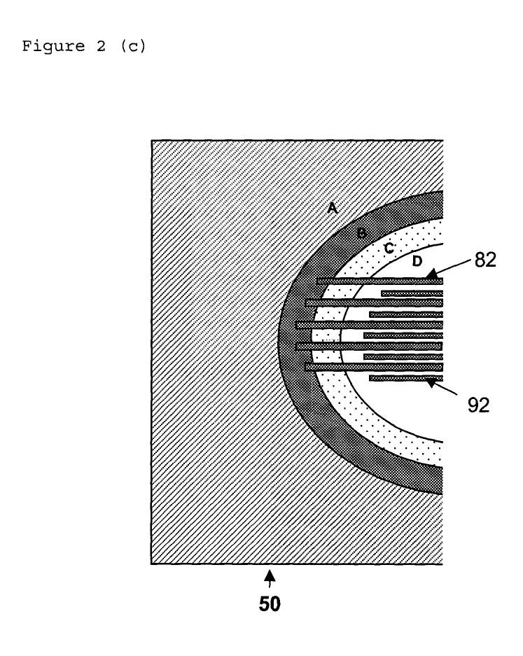

With particular reference to FIG. 2, a prototype EFS device 12 is shown. Silicone O-rings were glued to the device to form cell culture wells 50 and 60a, b, c. The EFS device had two basic electrode configurations to supply electric field stimulations to cells placed directly on the ITO electrodes on the bottom of the cell culture wells.

In the first electrode configuration (60a, b, c in FIG. 2a), the bottom surface of the well employed a single ITO electrode. These electrodes are available in a range of sizes and covered various portion of the surface area of the bottom of the wells. For example, in wells 60b and 60c, the oxynitride (SiOₓN_{y}) passivation layer was removed from the entire bottom surface of the well, leaving a 3500 μm diameter circle of ITO exposed to cells placed in well 60b and a 7000 μm diameter circle of ITO exposed in well 60c. However, in well 60a the oxynitride extended into the well such that only a 1750 μm diameter circle of ITO was exposed. The ITO surface electrodes in wells 60a-c were all connected to the contact point 72. The electrical circuit was formed via attaching a wire to connect contact point 72 and a second electrode made of Ag/AgCl inserted into the fluid bathing the cells in the wells. A voltage applied between these two electrodes created a vertical electric field capable of stimulating cells cultured inside these wells.

In the second electrode configuration (50 in FIG. 2a, b, and c), the bottom surface of the well comprised two ITO electrodes of opposing polarities (80 and 90 in FIG. 2b). These electrodes were fabricated as interdigitated by interleaved fingers (82 and 92 in FIG. 2b), which covered the entire surface of the well. While many widths and spacing of electrode fingers are possible in fabricating the interdigitated electrode fingers, the current EFS device employed 5 μm electrode fingers that were spaced 5 μm apart. In this configuration, a 20 μm diameter cell (100 in FIG. 2b) would contact at least two electrodes (one positive and one negative) resulting in efficient stimulation. Electrodes as small as 0.5 μm wide and 0.5 μm apart can be fabricated. Larger electrodes can also be produced. In well 50, one half of the interdigitated fingers extended from the right side of the well and were connected to contact point 72. The other half extended from the left side of the well and connected to the contact point 70. Thus, a voltage applied between contact 70 and 72 created a horizontal electric field capable of stimulating cells cultured on the bottom of this well. This electrode configuration removes the need for an electrode to be immersed into the well from above; thereby enabling easy access for fluidics and optical detection.

A magnified view of well 50 is shown in FIG. 2c. In region A of the well the glass substrate was covered with ITO capped with Ti/Pt and insulated with an oxynitride layer. In region B of the well, the Ti/Pt had been removed leaving ITO insulated with oxynitride. In regions C and D, the interdigitated fingers were formed by masking off areas of the transparent substrate during ITO deposition. The insulating layer of oxynitride was present in region C, but absent in region D, leaving the ITO interdigitated fingers directly exposed to the cells cultured on the bottom surface of the well. The perimeter of region D was also the approximate location of the adhesively bonded polymer cylinders (o-rings or cloning wells) that form the wells.

### EXAMPLE 3

### Coating the EFS Device With Factors to Promote Cell Attachment

Cells were seeded on the EFS device at a density of 1×10⁶ cells/ml and incubated at 37° C. Attachment of the cells to the bottom surface was determined 2 hrs after plating (cells should no longer be in suspension, or appear spherical). Incubation was continued for up to 16 hrs to allow attachment. If the cells remained in suspension after this 16 hr incubation, a factor to promote cell attachment was added to the EFS device. Exemplary factors included, but were not limited to, poly-d lysine, collagen or laminin. The concentration and composition of these factors required optimization for the cell type under investigation.

Coating wells in the EFS device with Collagen: Rat tail type I collagen stock (Cat No: C7661, Sigma-Aldrich, St. Louis Mo.) (3 mg/ml in 0.02 N acetic acid) was diluted to 50 μg/ml with 0.02 N acetic acid. Diluted collagen was added to the wells of the EFS device (100 μl in the large wells and 50 μl in the small wells) and incubated for 2 hrs at room temperature. After this, wells were washed 3 times with PBS and left to dry.

Coating wells in the EFS device with Poly-d-lysine: d-lysine stock (Cat No: P0899, Sigma-Aldrich, St. Louis Mo.) (3 μg/ml in PBS) was diluted to 0.3 μg/ml with 0.02 N acetic acid and added to wells of the EFS device (100 μl in the large wells and 50 μl in the small wells) and incubated for 2 h at room temperature. After this, wells were washed 3 times with PBS and left to dry.

### EXAMPLE 4

### Preparation of Cells for the EFS Device

This example illustrated the procedure for preparing SK-N-SH (ATCC HTB-11) human neuroblastoma cells for the EFS device. Similar procedures can be used to prepare other types of cells for the EFS device.

The SK-N-SH cells (ATCC HTB-11) were maintained in T-75 cell culture flasks at 37° C. (5% CO₂) in medium (20 ml) comprising Dulbecco's minimum essential medium (high glucose), adjusted to contain 10% (v/v) fetal bovine serum. All materials were obtained from Life Technologies unless otherwise stated.

Sub-culturing cells: Cell culture medium was removed from a flask containing a confluent monolayer of cells. The monolayer was gently washed with 5 ml PBS prior to addition of 2 ml 0.25% Trypsin/0.03% EDTA solution. The flask was incubated at 37° C. until the cells detached. 8 ml of cell culture medium was then added to the cell suspension and gently mixed. 2 ml of this mixture was added to a fresh T-75 flask containing 20 ml tissue culture medium and placed into a tissue culture incubator. Medium was replaced every 2-3 days until cells reach confluence.

Seeding EFS device: SK-N-SH cells did not require treatment of the EFS device with factors to promote cell attachment. However, coating of the EFS devices with poly-d-lysine or collagen, which is known to promote attachment of primary cells (such as neurons and hepatocytes to glass substrates) had no effect on subsequent electrical stimulation of SK-N-SH cells.

SK-N-SH cells were counted using a hemocytometer and their density adjusted to 1×10⁶ cells/ml with tissue culture medium. This cell suspension was added to the treated wells of the EFS device (100 μl in the large wells and 50 μl in the small wells). Cells were cultured on the EFS device for 12 h prior to experimental manipulation.

Loading of cells with optically detectable marker: Changes in cellular parameters in response to stimuli, such as ions (Ca²⁺, Na⁺, K⁺), pH, membrane potential, metabolites (cAMP, IP₃) or proteins can be monitored by the selection of an appropriate reporter system (probe and detection apparatus). In the case of proteins (to monitor translocation or trafficking in response to stimuli), this may require the construction and subsequent expression of an epitope tagged protein (this tag may consist of a fluorescent protein/peptide domain that is fused to the protein of interest). Monitoring changes in ions, pH, membrane potential and metabolites simply requires introducing the appropriate fluorescent dye into the cells (fura-2: Ca²⁺, SBFI: Na⁺, for example). The fluorescent properties of the dye changes as the parameter of interest fluctuates (a change in fluorescence intensity or a spectral shift in fluorescence may be observed). Selection of the dyes and conditions for introducing them into cells correctly depends on the cell type and the detection system used. Changes in plasma membrane potential were monitored in SK-N-SH cells using the membrane potential dye.

The dye was re-constituted (at 2× concentration) in physiological buffer comprising 25 mM Hepes, pH 7.4, 121 mM NaCl, 4.7 mM KCl, 1.2 mM KH₂PO₄, 1.2 mM MgSO₄, 5 mM NaHCO₃, 2 mM CaCl₂, 10 mM glucose and 0.25% (w/v) BSA. The dye was stored at −20° C. prior to use. On the day of the experiment, the membrane potential dye was diluted to 1× using physiological buffer. The cells on the EFS device were washed once with physiological buffer prior to addition of 1× membrane potential dye (100 μl in the large wells and 50 μl in the small wells). The cells were incubated for 1 h in 1× membrane potential dye at 37° C. prior to experimental manipulation.

ITO did not appear to affect normal cell physiology, as the presence of ITO affected neither cell viability nor activation of Na⁺ channels by veratridine (Table 1). SK-N-SH neuroblastoma cells were plated on glass-bottomed 96-well plates coated with ITO. Depolarization elicited by 30 μM veratridine was recorded using the Fluorimetric Imaging Plate Reader (FLIPR™). Cell viability was determined in parallel, using trypan blue. Data shown are the mean of three separate experiments (three wells per condition, per experiment). Parallel preparations of non-coated glass-bottomed plates were included as a control.

TABLE 1
Effects of ITO on tetrodotoxin sensitivity of veratridine-mediated
changes in membrane potential and cell viability

| Condition | Tetrodotoxin IC₅₀ (nM) | % Viability |
|---|---|---|
| ITO coated plates | 9.50 ± 0.15 | 98.05 ± 0.20 |
| Non-coated plates | 6.69 ± 0.29 | 99.00 ± 0.32 |

### EXAMPLE 5

### Imaging Changes in Plasma Membrane Potential in Single Living Cells

All materials were obtained from Sigma-Aldrich (St. Louis, Mo.) unless otherwise stated. The use of transparent metal oxide electrodes in the EFS device enabled the use of optical methods to detect changes in cellular parameters (via dyes/fluorescent proteins). The EFS device can be configured for use in any fluorescence-based instrument, potentially with any fluorescent probe.

All experiments were carried out at 37° C. in an atmosphere of 5% CO₂ (using the environmental chamber on the Pathway-HT). The choice of excitation and emission wavelengths to use depended on the fluorescent probe being used and was obtained from the product literature.

Membrane potential imaging experiments were performed in buffer, comprising of (in mM) 25 HEPES, 121 NaCl, 5 NaHCO₃, 10 glucose, 4.7 KCl, 1.2 KH₂PO₄, 1.2 MgSO₄, 1.5 CaCl₂ and 0.25% (w/v) fatty acid-free BSA (pH 7.4 @ 37° C.). SK-N-SH cells were seeded onto the EFS device in DMEM+10% (v/v) FBS. Following an overnight (12 h) incubation, cells were loaded with membrane potential dye for FLIPR (Molecular Devices, Sunnyvale Calif.) for 30 min according to the manufacturer's protocol. The cells were transferred to a thermostatically regulated microscope (Pathway-HT, Atto Biosciences, Rockville Md.). The EFS device was placed on a microscope with the wells uppermost and the conductive strip towards the front. The microscope objective was then brought into position from below. Silver wires were attached at the appropriate points on the conductive strip or immersion electrode, using small copper clips (depending on the well being tested). The wires were then connected to a Grass Telefactor S48 electrical stimulator via an SIU5 stimulus isolation unit (Grass-Telefactor/Astro-Med, Inc., West Warwick, R.I.). This apparatus was used to supply a range of electrical stimuli to the cells under observation.

Cells (dye-loaded) were visualized on the Pathway-HT using a 20× objective (Olympus). Fluorescence localized to the plasma membrane of cells was determined 1 h after loading the cells with dye, using excitation light of wavelength 488 nm (AttoArc light source, 50% illumination intensity). Emitted light was collected using a 510 nm long band pass filter and recorded using a 16 bit cooled charge-coupled device (CCD) camera. Fluorescence images were acquired every 500 ms and the fluorescence intensity at the plasma membrane from every cell within the field of view recorded (the image analysis software within the Pathway-HT program automatically focused on the cells and determined the appropriate areas to record fluorescence). Changes in fluorescence intensity, corresponding to changes in membrane potential were analyzed off-line using microsoft Excel.

Data analysis: The software controlling the Pathway-HT was able to automatically discern fluorescence from a specific probe/sub-cellular localization from non-specific fluorescence or background. The signal from the specific areas was termed a region of interest. These regions of interest could include single cells or regions within a single cell, depending on the application. The software exported the data from these regions of interest as a text file (as well as images). This text file contained data from all regions of interest over the duration of the whole experiment, which enabled changes in fluorescence to be quantified into changes in membrane potential (or another parameter) in response to external stimuli. Rate of change of fluorescence was calculated using linear regression, while maximum amplitude was calculated using the statistical functions in Microsoft excel. Data from all regions of interest in a given well were pooled and the average values calculated (+/−SD).

### EXAMPLE 6

### Optimization of Electrical Stimulation Protocols

The electrical stimulation must be optimized for each electrode configuration, and cell line and physiological response of interest. Voltage amplitudes/durations that result in cell death (observed as detachment from the electrode surface) or heating of the medium bathing the cells must be avoided.

Use of the Grass Telefactor electrical stimulator enabled the application of a wide variety of electrical stimuli to cells cultured on the EFS device. Variation of the voltage, duration, number and current profile of the stimuli were possible (as well as the orientation of the resulting electric field due to electrode configuration). Once the electric field reached a threshold potential, the physical properties of a voltage-gated ion channel in the plasma membrane of the cells would change (they may change from an inactive configuration to active; or their conductive state may change). These changes would induce changes in membrane potential and could be optically measured using electrochromic transmembrane potential dyes.

Changes in plasma membrane potential, cell position and temperature of the medium were recorded. Observed changes in membrane potential under different electrical stimulation protocols in single cells were normalized to the response observed following maximal depolarization with 50 mM KCl treatment.

i) Pulse duration: A range of voltage stimuli, ranging from 2 to 120 V, were applied to the EFS, changes in membrane potential and medium temperature were recorded. Pulse durations were varied at all voltages tested. Pulse durations that did not evoke changes in membrane potential, or resulted in cellular detachment/medium heating were rejected. The pulse duration that evoked maximal depolarization was chosen.

ii) Voltage amplitude: Using the pulse duration that gave maximal depolarization, the voltage amplitude was varied from 0V to maximum (that which did not result in cell detachment). The voltage that evoked half-max depolarization was chosen, so that the action of compounds that increase or decrease cellular activity in response to voltage could be investigated.

iii) Number of pulses: Using the pulse length and voltage parameters determined above, the number of pulses in each stimuli were varied to maximize cellular response. Multiple pulses that resulted in detachment and heating were rejected.

The use of an electrical stimulus that evoked a half maximal response under normal conditions would give an acceptable “dynamic range” in which decreases or increases in the response could be observed. It was found that the optimal stimulation parameters for SK-N-SH cells to be a series of square-wave voltage pulses (750 μs pulses at 8 pulses/s for 3 s), wherein the voltage was stepped from 0 to a positive voltage, preferably less than 120 V.

### EXAMPLE 7

### Electrical Stimuli Induced Changes in Plasma Membrane Potential of SK-N-SH Cells

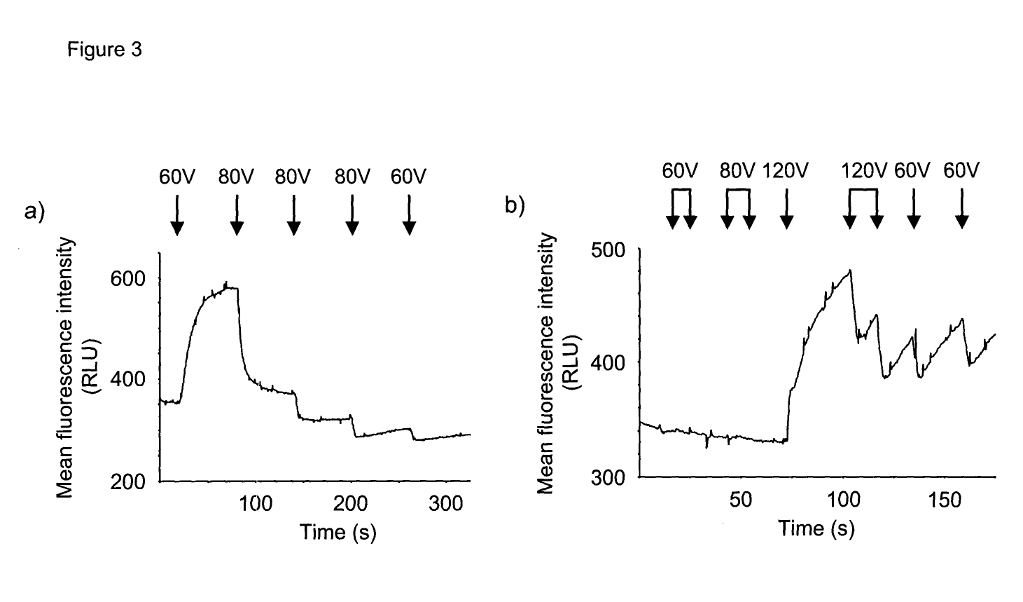

Voltage sensitive dye fluorescence from individual SK-N-SH cells was monitored using a digital fluorescent microscope (Pathway-HT, Atto Bioscience, Rockville Md.) over the course of 10 minutes. Trains of electrical pulses (square wave pulse of 750 μs duration, at 8 pulses/s for 3 s) were applied to the EFS device at the times and voltages indicated (FIG. 3) from the user-controlled output of an electronic stimulator (Grass model S48, Grass-Telefactor/Astro-Med, Inc., West Warwick, R.I.). Voltage pulses were applied to the cells either via interdigitated electrode fingers (IDF, FIG. 3a), or solid ITO surface and an immersed Ag/AgCl electrode (FIG. 3b).

Analysis of the images showed that in any given well cells responded homogeneously to the applied electric field stimuli. The mean fluorescence of cells cultured on IDF electrodes increased (corresponding to depolarization of the plasma membrane) from a basal value of 291.47±2.78 to a maximum of 580.17±8.92 relative fluorescence units (RFU) following application of trains of electric field stimuli of 80 V amplitude (FIG. 3a). However, larger voltages were required to evoke responses from cells cultured on the solid electrode as compared to cells grown on IDF electrodes. The mean fluorescence of cells cultured on solid ITO electrodes increased from a basal value of 325.30±3.14 to a maximum of 479.15±9.40 relative fluorescence units (RFU) following application of an electric field stimuli train of 120 V amplitude (FIG. 3b). Similar responses were observed in studies carried out using other devices, where vertical as well as horizontal electric fields were applied to cells (WO200208748). These findings may be a result of current “leakage” through gaps in the cell monolayer. The effect of cell density on current leakage, together with the susceptibility of Ag/AgCl electrodes to poisoning, led to a decrease reproducibility of electric field-induced responses in experiments where an immersion electrode was used.

Decreases in fluorescence were observed at the highest voltages (≧80 V for IDF electrodes and>120 V for Ag/AgCl immersion electrodes). Analysis of the digital images obtained showed that the decreases in fluorescence were not due to repolarization of the plasma membrane, but instead due to detachment of the cells from the electrode surface. These findings highlighted the importance of the acquisition of spatial as well as temporal information in the testing of the EFS prototype.

The data shown in FIG. 3 suggests that IDF electrodes were able to induce plasma membrane depolarization in SK-N-SH cells at lower voltages than the solid electrode configuration. Another drawback of the immersed solid electrode configuration was the difficulty in incorporating fluidics to introduce solutions to each well due to the presence of the Ag/AgCl electrode that was inserted into the well from above. Furthermore, while data taken from IDF electrodes was highly reproducible, data taken from cells stimulated with the solid electrode varied greatly, depending on cell confluency. In addition, it was found that SK-N-SH cells oriented themselves along the IDF electrodes, which possibly resulted in a more efficient electrical circuit. Therefore, the IDF electrode configuration is the favored configuration among the two described herein.

### EXAMPLE 8

### Role of Voltage-Gated Sodium Channels in Voltage-Induced Plasma Membrane Depolarization in SK-N-SH Cells

SK-N-SH cells express a mixed population of ion channels on the plasma membrane, including the voltage-gated sodium channel. Therefore, the changes in plasma membrane potential observed could be a summation of the alteration in activity of this heterogeneous population of ion channels. Experiments were performed to ascertain the role of the flux of Na⁺ through the voltage-gated sodium channels in voltage-induced plasma membrane depolarization in SK-N-SH cells.

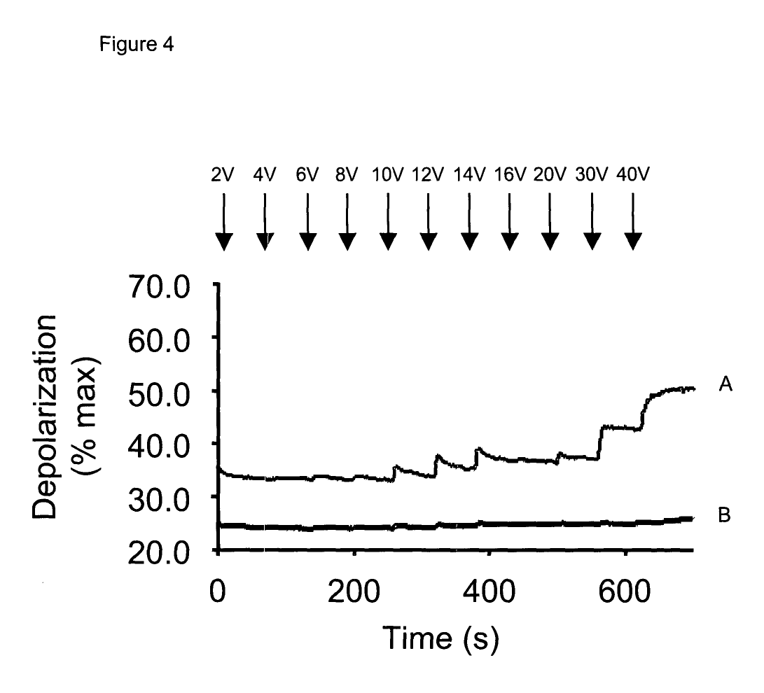

Increases in fluorescence were observed in all SK-N-SH cells cultured on IDF electrodes, in response to stimuli trains up to 40V amplitude (FIG. 4, curve A). The fluorescence increases were likely due to depolarization, the amplitude and rate of which varied in a voltage-dependent manner. Treatment of the cells with the selective Na⁺ channel inhibitor tetrodotoxin (TTX) at a concentration of 100 nM, completely abolished voltage-induced changes in plasma membrane potential (FIG. 4, curve B). During the assay, TTX was either added to the cells during the reporter dye incubation period or added immediately prior to, or following electrical stimulation. In addition, removal of extracellular Na⁺ abolished electric field-mediated changes in membrane potential. These data suggest that voltage-gated sodium channels are responsible, in part, for the observed changes in plasma membrane potential.

Although the maximum depolarization of SK-N-SH cells was observed following a 40V stimuli train, there was a change in the kinetics of the observed responses evoked from stimuli trains of amplitudes greater than 14 V (FIG. 4). Stimulus trains of 16-20 V did not cause significant increases in membrane potential (over that already induced under the experimental conditions). At amplitudes 20 V and above, the observed depolarizations appeared to be more sustained than those observed at lower voltages. These data may be due to several factors. Firstly, repeated stimulation may result in inactivation of the ion channels, which requires stimuli trains 20 V or greater to overcome. Secondly, higher voltages may activate other families of voltage-gated ion channels. Thirdly, higher voltage trains may evoke non-physiological responses, which may subsequently result in cellular detachment.

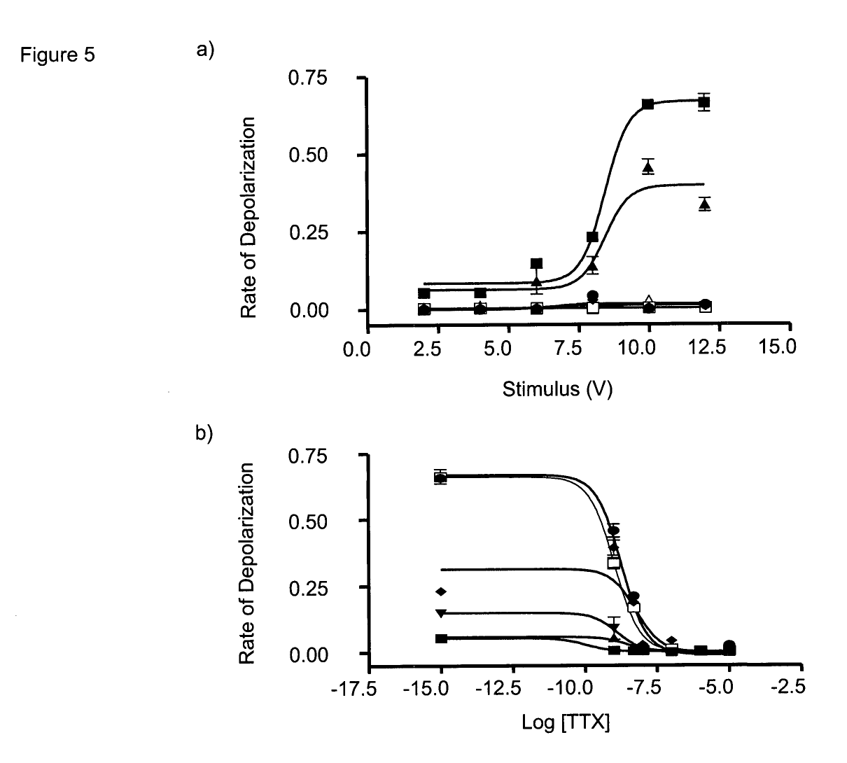

In these experiments, the calculated EC₅₀ for voltage amplitude was found to be approximately 8 V (FIG. 5a). Every cell in a field of view responded and the responses were similar from cell to cell (the data are mean +/−SD for 100 cells from 3 separate experiments) (control, CTRL, FIG. 5a). TTX blocked the depolarizations in a dose-dependent manner. The IC₅₀ for TTX was found to be approximately 9 nM (FIG. 5b), which was in agreement with previous studies carried out using conventional techniques (for review, see Clare et al., 2000, Drug Discovery Today, 5: 506-520).

To ascertain if other channels participated in the observed changes in plasma membrane potential, studies were carried out as per the conditions described in FIG. 5, using a variety of pharmacological agents that block voltage-gated Ca²⁺ or voltage- and Ca²⁺-activated K⁺ channels (Table 2). Electrical stimuli were applied to cells cultured on the EFS device. Cells were pre-incubated for 30 min with the inhibitors at the concentrations shown. Each stimulus consisted of a train of 8 V 750 μs pulses (8 pulses per s, for 3 s). Data shown is the mean of three separate experimental preparations (100 cells per preparation). Results are expressed as a percentage of the maximal depolarization observed in control cells with 50 mM KCl (100 cells per preparation). No significant effect of these inhibitors on the cells' response to applied electrical stimuli was observed (Table 2).

TABLE 2
Effects of inhibitors of various voltage-gated and Ca²⁺-
activated ion channels on EFS voltage-evoked
increases in membrane potential of SK—N—SH cells

| Conditions | Rate (% change/ s) | Amplitude (% max) |
|---|---|---|
| Non-stimulated | 0.00 ± 0.000 | 24.510 ± 0.370 |
| Control | 0.120 ± 0.003 | 34.090 ± 0.320 |
| TTX (100 nM) | 0.001 ± 0.001 | 24.260 ± 0.290 |
| Verapamil (100 μM) | 0.141 ± 0.014 | 32.84 ± 0.330 |
| Clotrimazole (1 μM) | 0.124 ± 0.024 | 33.92 ± 0.320 |
| Apamin (100 nM) | 0.114 ± 0.012 | 35.67 ± 0.641 |
| Charybdotoxin (100 nM) | 0.115 ± 0.013 | 34.09 ± 0.326 |

These data suggest that activation of voltage-gated sodium channels and subsequent influx of sodium across the plasma membrane played a primary role for the observed changes in plasma membrane potential in response to electric field stimulation.

### EXAMPLE 9

### Activation of Voltage-Gated K⁺ Channels Using the EFS

Examples described supra show that the EFS device can be used to evoke voltage-dependant activation of the voltage-gated Na⁺ channel. In this example, the ability of the EFS device to alter the activity of other voltage-gated ion channels, such as voltage-gated K⁺ channels was evaluated, and the potential of using the EFS device in a screen for inhibitors of such channels was also tested.

Over expression of hERG channels in HEK cells has previously been shown to enhance KCl-induced plasma membrane depolarization as compared to parental cells (Taylor, B: Molecular Devices Users Meeting presentation, May 21-25, 2002). In addition, removal of Na⁺ in the growth medium has been reported to destabilize hERG inactivation (Numaguchi et al 2000, Circ. Res. 87: 1012-1018). Verification experiments were first performed to show that the recombinant HEK cells to be used in the EFS device assays stably over-expressed hERG channels. Dose-dependant plasma membrane depolarization in response to increased KCl concentration in the growth medium was observed in these recombinant cells. The amplitude and rate of the observed depolarization was reduced by the removal of extracellular Na⁺ concentration. The observed depolarization was also reduced by exposing the recombinant cells to cisapride (Taylor, B: Molecular Devices Users Meeting presentation, May 21-25, 2002), an inhibitor for hERG channels. This reduction effect was more pronounced in the absence of extracellular Na⁺ and in cells that were not pre-incubated with the compound. These data suggest that these recombinant HEK cells indeed express functional hERG channels.

Recombinant HEK cells stably over-expressing the hERG channel were cultured on the EFS device and subjected to a range of voltage stimuli (750 μs pulses at 8 pulses/s for 3 s, the amplitude and times are noted below). Changes in plasma membrane potential were recorded using the Pathway-HT. The ability of both the solid and interdigitated electrode configurations to supply stimuli to the cells was tested. The effect of extracellular Na⁺ on hERG conductance was also examined with the EFS device.

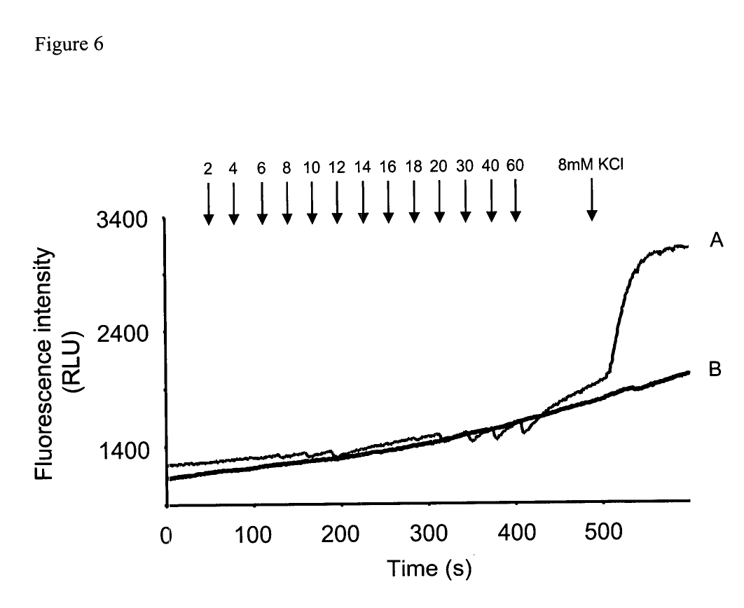

As shown in FIG. 6, voltage stimuli evoked changes in plasma membrane potential of HEK cells stably expressing hERG channels when cells were cultured in a well of the EFS containing the interdigitated electrode. Application of voltage stimuli resulted in hyperpolarization of the plasma membrane. The amplitude and rate of the hyperpolarization appeared to be dose-dependant on the voltage stimuli. Pre-incubation of the cells with cisapride blocked the cells' response to voltage. These data suggested that the observed responses were due to voltage dependant activation of hERG.

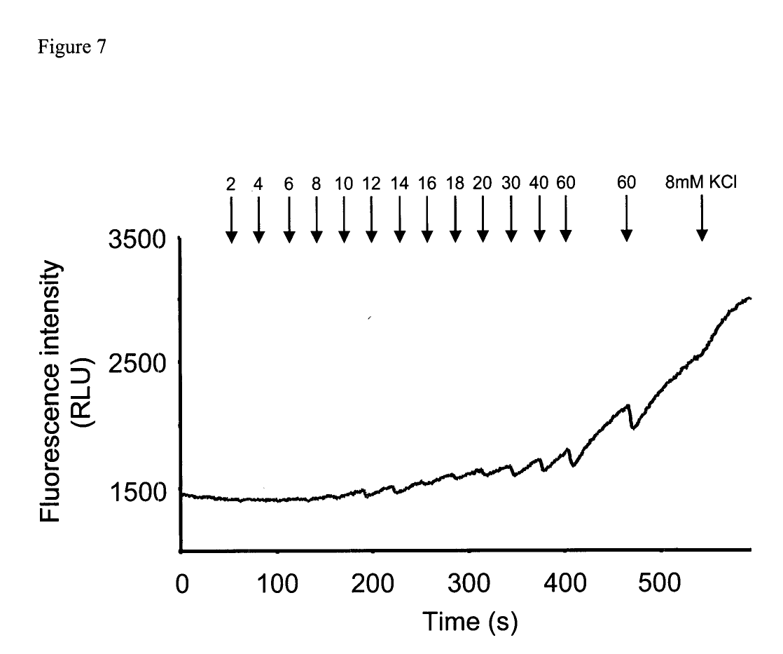

Hyperpolarization of plasma membranes of HEK cells stably expressing hERG channels was also observed when cells were stimulated with electric field applied through a transparent electrode and a solid electrode made of Ag/AgCl electrode (FIG. 7), not the interdigitated electrodes. Voltages applied from both electrode configurations stimulated cells similarly and resulted in similar hyperpolarization of the cell membranes.

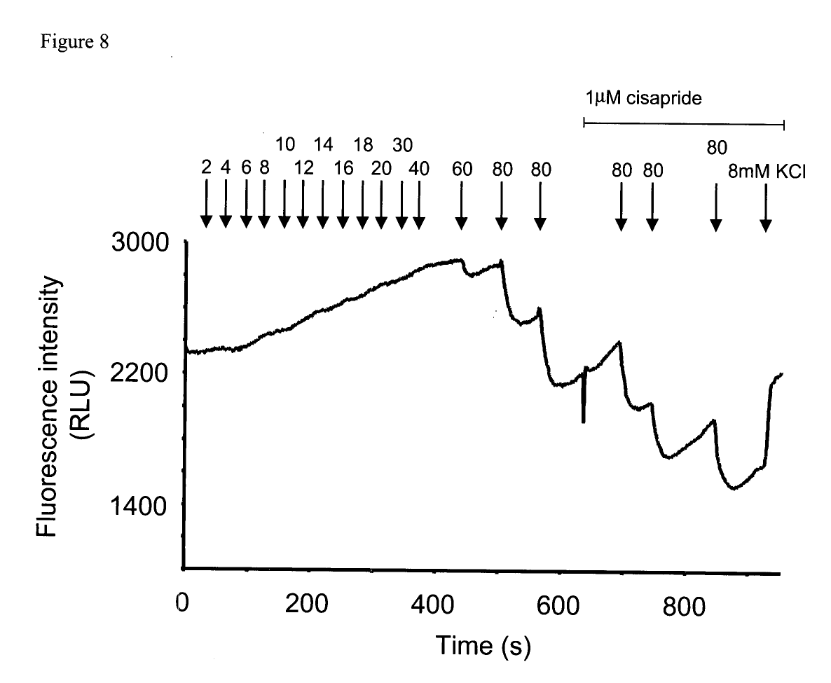

Removal of extracellular Na⁺ appeared to shift the observed voltage-response relationship right-ward (FIG. 8).

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method of measuring a physiological response in a cell, comprising the steps of:
1) introducing one or more cells in a liquid medium into a well of an electric field stimulation device, wherein said device comprising at least one transparent electrode disposed on the surface of the transparent bottom of the well;
2) labeling said cell with an optically detectable marker;
3) exposing said cell to repetitive electric pulses supplied by the transparent electrode and a second electrode of opposing polarity, wherein said repetitive electric pulses are of about between 250-1000 μs duration at about 1-100 pulses/s and about 2-120 V amplitude, wherein said electric pulses produce a controlled change in a physiological response of said cell;
4) detecting an optical signal associated with the optically detectable marker; and
5) comparing the optical signal measured in step (4) with an optical signal measured from a cell that is not exposed to the repetitive electric pulses.

2. The method of claim 1, wherein said physiological response in a cell comprises a change in the activity of an ion channel, a change in the secretion or absorption of a biological molecule by the cell, plasma membrane rearrangement, intracellular rearrangement, a change in cellular metabolism, apoptosis, or gene transcription.

3. A method of characterizing the biological activity of a candidate compound, comprising the steps of:
1) introducing one or more cells in a liquid medium into a well of an electric field stimulation device, wherein said device comprising at least one transparent electrode disposed on the surface of the transparent bottom of the well;
2) labeling said cell with an optically detectable marker;
3) contacting the cell with a test compound;
4) exposing said cell to repetitive electric pulses supplied by the transparent electrode and a second electrode of opposing polarity, wherein said repetitive electric pulses are of about 250 to about 1000 μs duration at about 1 to about 100 pulses/s and about 2 to about 120 V amplitude, and produce a controlled change in a physiological response of said cell;
5) detecting an optical signal associated with the optically detectable marker; and
6) comparing the optical signal measured from step 5) with an optical signal measured from a cell that is not contacted with the candidate compound.

4. The method of claim 3, wherein said transparent electrode comprises an electrically conductive transparent material or is a metallic optically transparent electrode.

5. The method of claim 4, wherein said electrically conductive transparent material is selected from a group consisting of indium tin oxide (ITO), zinc oxide (ZnO), SnO2, CdO, MgIn2O4, Al-doped ZnO film, a diamond thin film, and a combination thereof.

6. The method of claim 5, wherein said electrically conductive transparent material is indium tin oxide (ITO).

7. The method of claim 4, wherein said transparent electrode further comprises a layer of an insulating transparent material external to the electrically conductive transparent material.

8. The method of claim 7, wherein said insulating transparent material is a transparent dielectric, selected from silicon dioxide (SiO2), silicon nitride (Si3N4), and silicon oxynitride (SiOxNy).

9. The method of claim 7, wherein the thickness of the insulating transparent material is about 100 Å to about 2000 Å.

10. The method of claim 3, wherein said second electrode of opposing polarity is inserted into the fluid bathing the cells inside the well, wherein a voltage applied between the transparent electrode and the second electrode creates a vertical electric field capable of stimulating cells inside the well.

11. The method of claim 10, wherein said second electrode of opposing polarity comprises an electrically conductive transparent material or is a metallic optically transparent electrode.

12. The method of claim 10, wherein said second electrode comprises an electrically conductive non-transparent material.

13. The method of claim 12, wherein said electrically conductive non-transparent material is selected from the group consisting of gold, platinum, palladium, chromium, molybdenum, iridium, tungsten, tantalum, titanium, stainless steel, carbon, graphite and polypyrrole.

14. The method of claim 3, wherein the electric field stimulation device comprises two transparent electrodes with opposite polarity that are disposed on the surface of the transparent bottom of the well, wherein a voltage applied between the two transparent electrodes creates a horizontal electric field capable of stimulating cells inside the well.

15. The method of claim 14, wherein the two transparent electrodes are fabricated to contain interdigitated fingers covering the surface of the transparent bottom of the well.

16. The method of claim 15, wherein the interdigitated fingers include a width and spacing such that a single cell can contact at least two or more electrodes of opposing polarity.

17. The method of claim 3, wherein said physiological response is a change in the conductivity of an ion channel wherein the ion channel is selected from the group consisting of a potassium channel, a calcium channel, a chloride channel, a sodium channel, a non-specific ion channels, and a combination thereof.

18. The method of claim 17, wherein said physiological response is a change in the conductivity of a voltage-gated ion channel.

19. The method of claim 3, wherein said optically detectable marker is a fluorescent dye, a radioactive ion, a fluorescent protein, a luminescent protein, a protein tagged with a fluorescent or luminescent epitope, or a change in the refractive index of the cells.

20. The method of claim 3, wherein said optically detectable marker is a voltage sensor selected from the group consisting of FRET based voltage sensors, electrochromic transmembrane potential dyes, transmembrane potential redistribution dyes, radioactive ions, ion sensitive fluorescent or luminescent dyes, and ion sensitive fluorescent or luminescent proteins.

21. The method of claim 3, wherein said cell is a eukaryotic cell.

22. The method of claim 3, wherein said cell is a prokaryotic cell.

23. The method of claim 3, wherein said cell is associated with a biological tissue.

24. The method of claim 3, wherein the optical signal associated with the optically detectable marker is monitored via an imaging system.

25. The method of claim 24, wherein the imaging system comprises a microscope connected to a charge-coupled device camera, a photodiode array, or a photomultiplier tube.

26. The method of claim 24, wherein the imaging system comprises a plate reader, connected to a charge-coupled device camera, a photodiode array, or a photomultiplier tube.

27. The method of claim 3, wherein said repetitive electric pulses are supplied in a square wave-form, a sinusoidal wave-form, or a saw tooth wave-form.

28. The method of claim 27, wherein said repetitive electric pulses are supplied in a square wave-form.

29. The method of claim 28, wherein said repetitive electric pulses have an amplitude within the range of about 20 to about 100 V.

30. The method of claim 28, wherein said repetitive electric pulses have a pulse duration within the range of about 250 to about 1000 per pulse.

31. The method of claim 30, wherein said repetitive electric pulses are supplied to the cell in about 750 μs per pulse at about 8 pulses per second, and the train of pulses lasts about 3 seconds.

32. The method of claim 3, further comprising the step of coating the surface of the transparent bottom of the well with a factor to promote cell attachment.

33. The method of claim 32, wherein the factor is selected from poly-d-lysine, poly-l-lysine, collagen type 1, collagen type IV, heparin sulphate proteoglycan, laminin, fibronectin, vitronectin, gelatin or poly-l-ornithine.

34. A system for supplying electric field stimulation to a cell and optically monitoring a physiological response of the stimulated cell, comprising
1) an electric field stimulation device comprising a well and an transparent electrode disposed on the surface of the transparent bottom of the well;
2) a cell labeled with an optically detectable marker placed and its bathing fluid within the well of the electric field stimulation device;
3) a means for providing electrical stimulation; and
4) an imaging system for detecting the optical signal from the cell.
